(19) United States
(12) Patent Application Publication
(10) Pub. No.: US 2022/0333186 A1
(43) Pub. Date: Oct. 20, 2022

(54) METHOD AND SYSTEM FOR TARGETED NUCLEIC ACID SEQUENCING

(71) Applicant: JUMPCODE GENOMICS, INC., San Diego, CA (US)

(72) Inventor: Keith BROWN, Carlsbad, CA (US)

(21) Appl. No.: 17/761,866

(22) PCT Filed: Sep. 25, 2020
(86) PCT No.: PCT/US2020/052652

§ 371 Date: Mar. 18, 2022

Related U.S. Application Data
(60) Provisional application No. 62/906,636, filed on Sep. 26, 2019.

(51) Int. Cl. C12Q 1/6855; C12Q 1/6806

(52) U.S. Cl. CPC C12Q 1/6855; C12Q 1/6806; C12Q 1/6869

(57) ABSTRACT

Disclosed herein are methods, compositions, and systems that utilize the consensus sequencing of rolling circle amplified short templates. Methods of determining a nucleic acid sequence can include one or more steps of contacting a nucleic acid in a sample to an endonuclease to cleave a target nucleic acid; ligating the target nucleic acid sequence to form a circular target nucleic acid; hybridizing at least one primer to the circular target molecule to form amplified nucleic acid through rolling circle amplification; and performing sequence analysis of the amplified nucleic acid.

### CROSS-REFERENCE

This application claims the benefit of U.S. Provisional Application No. 62/906,636, filed Sep. 26, 2019, which is incorporated herein by reference in its entirety.

### BACKGROUND

The disclosure herein relates to the field of molecular biology, such as amplification and identification of nucleic acid sequence through consensus sequencing of rolling circle amplified template and method of preparing such template.

PCR, or variants of PCR technology along with hybrid capture are dominant methods of targeted sequencing. Although widely used, both have limitations for long read sequencers. Hybrid capture uses short RNA or DNA probes with biotin to hybridize to target DNA and “pull down” the sequences of interest. For long target sequences, this approach is inefficient, both because many oligonucleotide probes are required and because the process often results in physical shearing of the long DNA molecules during the pulldown process. These defects limit the length of the contiguous sequencer read using single molecule technologies.

Long range PCR has been used as an alternative, but also presents challenges. For example, long range PCR is hard to multiplex. Often, one loses the ability to detect large chromosomal events such as translocations due to the requirement of opposing PCR primers on opposite strands outside of the target region. In addition, the clonal amplification of PCR limits sensitivity to detect low frequency somatic variation in a heterogeneous sample such as a tumor, and may propagate polymerase errors such as point mutations or translocations from the early cycles of the reaction. Further, long-range PCR sometimes exhibits template switching, creating errors in the amplification product. Therefore, there is a need for developing a more viable method for sequencing a nucleic acid.

### SUMMARY

Provided herein are methods, compositions, and systems that utilize the consensus sequencing of rolling circle amplified short templates and can produce highly accurate consensus sequencing of the short read sequencers with the speed and portability of long read sequencers. The methods and compositions are applicable to biological, clinical, forensic, and environmental samples. Methods of determining a nucleic acid sequence can include one or more steps of contacting a nucleic acid in a sample to an endonuclease to cleave a target nucleic acid; ligating the target nucleic acid sequence to form a circular target nucleic acid; hybridizing at least one primer to the circular target molecule to form amplified nucleic acid through rolling circle amplification; and performing sequence analysis of the amplified nucleic acid. The sequencing methods and systems described herein provide several advantages over traditional targeted sequencing application, including a faster time to result with greater access to data in both low budget laboratories as well as in the field; less bias during amplification; great compatibility with highly degraded templates; and reduced polymerase errors using target specific (junction) primers during the rolling cycle application.

Provided herein, in certain aspects, are methods of determining a nucleic acid sequence. In some cases, methods comprise: contacting a nucleic acid in a sample to an endonuclease to cleave a target nucleic acid; ligating the target nucleic acid to form a circular target nucleic acid; hybridizing at least one primer to the circular target nucleic acid; amplifying the circular target nucleic acid through rolling circle amplification to generate an amplified nucleic acid; and performing a sequence analysis of the amplified nucleic acid. In some cases, the target nucleic acid comprises at least one of a Clustered Regularly Interspaced Short Palindromic Repeats (CRISPR) sequence, a zinc finger nuclease (ZFN) sequence, and a transcription activator-like effector nucleases (TALENs) sequence. In some cases, the endonuclease is a restriction enzyme specific to at least one site on the nucleic acid. In some cases, the endonuclease comprises at least one selected from Clustered Regulatory Interspaced Short Palindromic Repeat (CRISPR)/Cas system protein-gRNA complex, Zinc Finger Nucleases (ZFN), and Transcription activator like effector nucleases (TALEN). In some cases, the endonuclease is a Clustered Regulatory Interspaced Short Palindromic Repeat (CRISPR)/Cas system protein-gRNA complex, and wherein the gRNAs are complementary to at least one site on the nucleic acid. In some cases, the primer is selected from random primer, locus specific primer, or a combination thereof. In some cases, the endonuclease cleaves the nucleic acid to form a 5′ end or a 3′ end of the target nucleic acid. In some cases, the method comprises hybridizing a first primer to the circular target nucleic acid, wherein a first primer comprises at least one sequence complementary to at least a portion of the 5′ end and the 3′ end of the target nucleic acid. In some cases, the method comprises hybridizing a first primer and a second primer to the circular target nucleic acid, wherein a first primer comprises at least one sequence complementary to at least a portion of the 5′ end and the 3′ end of the target nucleic acid, and wherein a second primer comprises a sequence that is complementary to a portion of the target nucleic acid that is not adjacent to the 5′ end or the 3′ end. In some cases, performing a sequence analysis of the amplified nucleic acid comprises performing a nanopore sequencing analysis. In some cases, the primer comprises an adaptor that binds to a motor protein used in the nanopore sequence analysis. In some cases, the amplifying and the sequencing analysis are performed concurrently. In some cases, the amplifying and the sequencing analysis are performed concurrently, and wherein the sequencing analysis is performed until a pre-determined minimum required accuracy of a template is achieved. In some cases, the method comprises contacting the sample with exonuclease after step (b). In some cases, the exonuclease does not degrade the circular target nucleic acid. In some cases, the method comprises performing rolling circle amplification in situ on the circular target nucleic acid. In some cases, the exonuclease is S1 nuclease. In some cases, the nucleic acid comprise any one of single stranded DNA, double stranded DNA, single stranded RNA, double stranded RNA, cDNA, synthetic DNA, artificial DNA, and DNA/RNA hybrids. In some cases, the method comprises sequencing the amplified nucleic acid through a next-generation sequencing method. In some cases, the sample wherein the sample is selected from saliva, blood, plasma, serum, mucous, feces, urine, cerebrospinal fluid (CSF), skin, tissue, and bone. In some cases, ligating the target nucleic acid is performed using a CircLigase™. In some cases, ligating the target nucleic acid is performed using a single stranded splint, wherein the splint comprises sequences complementary to the ends of the target nucleic acid.

In another aspect, there are provided methods of preparing a nucleic acid for sequencing. In some cases, such methods comprise contacting a nucleic acid in a sample to an endonuclease to cleave a target nucleic acid; ligating the target nucleic acid sequence to form a circular target nucleic acid; hybridizing at least one primer to the circular target nucleic acid; and amplifying the circular target nucleic acid through rolling circle amplification. In some cases, the nucleic acid comprises at least one of a Clustered Regularly Interspaced Short Palindromic Repeats (CRISPR) sequence, a zinc finger nuclease (ZFN) sequence, and a transcription activator-like effector nucleases (TALENs) sequence. In some cases, the endonuclease is a restriction enzyme specific to at least one site on the nucleic acid. In some cases, the endonuclease comprises at least one selected from Clustered Regulatory Interspaced Short Palindromic Repeat (CRISPR)/Cas system protein-gRNA complex, Zinc Finger Nucleases (ZFN), and Transcription activator like effector nucleases. In some cases, the endonuclease is a Clustered Regulatory Interspaced Short Palindromic Repeat (CRISPR)/Cas system protein-gRNA complex, and wherein the gRNAs are complementary to at least one site on the nucleic acid. In some cases, the primer is selected from random primer, locus specific primer, or combinations thereof. In some cases, the endonuclease cleaves the nucleic acid to form a 5′ end or a 3′ end of the target nucleic acid. In some cases, the method comprises hybridizing a first primer to the circular target nucleic acid, wherein a first primer comprises at least one sequence complementary to at least a portion of the 5′ end and the 3′ end of the target nucleic acid. In some cases, the method comprises hybridizing a first primer and a second primer to the circular target nucleic acid, wherein a first primer comprises at least one sequence complementary to at least a portion of the 5′ end and the 3′ end of the target nucleic acid, and wherein a second primer comprises a sequence that is complementary to a portion of the target nucleic acid that is not adjacent to the 5′ end or the 3′ end.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

Reference is made to the following detailed description that sets forth illustrative embodiments, in which the principles of the invention are utilized, and the accompanying drawings of which:

FIG. 1 shows an exemplified scheme of the step of preparing a target nucleic acid.

FIG. 2 illustrates an exemplified scheme of the rolling circle amplification step.

FIG. 3 illustrates the primer and adaptor scheme used in the sequence analysis step.

FIG. 4 shows a CRISPR scheme used for cleaving the nucleic acid

### DETAILED DESCRIPTION

Disclosed herein are methods, systems, and compositions for sequencing technology that can provide for point of care detection of time critical genomic information. The methods and systems described herein allow for infectious disease detection in humans, plants and animals in real time and in remote locations, as well as forensic DNA analysis and microbiome detection. The sequencing methods and systems utilize the consensus sequencing of the rolling circle amplified (RCA) short templates and nanopore sequencing techniques to provide a faster time to result with greater access to data in both low budget laboratories as well as in the field and less bias during amplification. The methods and systems work well with highly degraded templates and reduces polymerase errors using target specific (junction) primers during RCA. The amplification step described herein only amplifies copies of the template molecule and avoid the problem of amplifying copies of copies that will propagate polymerase errors during the first or early amplification cycles of a technique like PCR.

Advances in genome sequencing technologies have greatly increased our understanding of human genetic variation and its contribution to disease. Short read DNA sequencing technologies (Illumina, Thermo Fisher, Qiagen) produce billions of short reads resulting in the routine identification of single nucleotide polymorphisms and small insertions and deletions. These short read sequencing technologies have not shown a sensitivity to detect more complex variation such as large scale chromosomal rearrangements, translocations and mobile element rearrangements. These systems are also often expensive and require 24 hours or more to complete a sequencing run. Long read sequencing technologies (Pacific Biosciences, Oxford Nanopore) have shown the ability to generate single molecule read lengths in excess of 10,000 base pairs, but do not have the capacity to sequence and assemble a full human genome. Sequencing strategies disclosed herein can produce highly accurate consensus sequencing of the short read sequencers with the speed and portability of long read sequencers.

The disclosure herein relates to sequencing methods and systems that can produce highly accurate consensus sequencing with fast read speed and great portability. Some embodiments relate to methods of consensus sequencing of rolling circle amplified short templates and method of preparing such templates.

### Sequencing of Targeted Nucleic Acids

Methods and uses of the compositions disclosed herein allow one to determine the sequence at any targeted site in a genome or other nucleic acid sample, including repetitive elements as well as average complexity DNA sequences, for example mRNA coding sequences. Accordingly, methods herein can be applied to any desired location in the genome, or to other repetitive or non-repetitive nucleic acid samples.

Methods of determining a nucleic acid sequence can include one or more steps of contacting a nucleic acid in a sample to an endonuclease to cleave a target nucleic acid; ligating the target nucleic acid sequence to form a circular target nucleic acid; hybridizing at least one primer to the circular target molecule to form amplified nucleic acid through rolling circle amplification; and performing sequence analysis of the amplified nucleic acid.

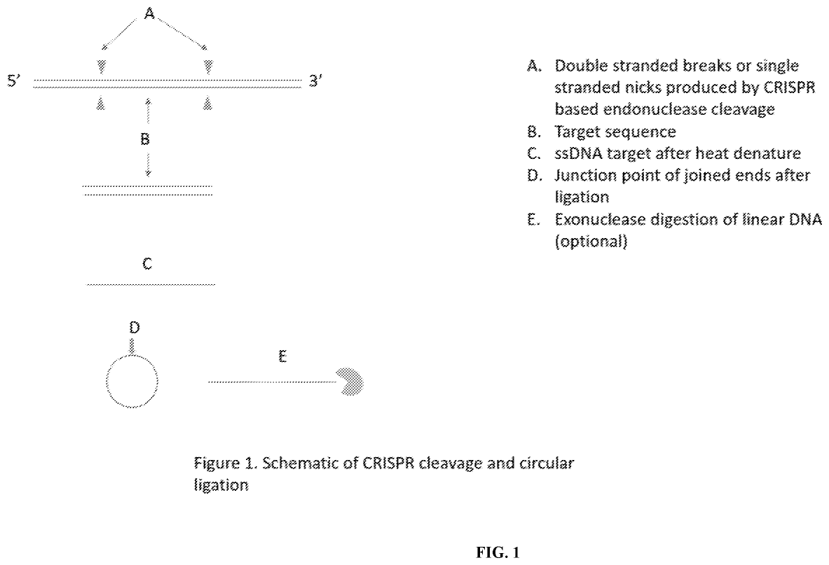

A number of workflows are consistent with the disclosure herein. Representative workflows are as follows, although variants are also contemplated. FIGS. 1 to 3 provide illustrations of the workflow involved in the various steps of the sequencing methods.

FIG. 1 provides a non-limiting exemplified illustration of the cleavage and circularization steps. First, a nucleic acid is obtained through extraction and purification or from crude samples such as directly from blood, serum, plasma, tears, saliva, urine, fecal, tissue, plant, soil or water, or other sample source. At A, in-vitro CRISPR-CAS systems or similar derivatives are used to produce a nick or double stranded cut flanking a region of interest or target sequence, shown at B, such as a segment of genomic DNA. This step may be applied to one target locus, such as 16s rRNA genomic sequence, or to many known genomic loci simultaneously, such as short tandem repeats (STRs) for forensic analysis, or to nongenomic targets such as reverse-transcribed cDNA targets from a transcriptome or other RNA sample, such as microRNA. Guide RNA (gRNA) sequences used for such steps are, in some cases, further than 30 bp from each other. The genomic material is then heat denatured to create single a stranded DNA target sequence shown at C and the shorter fragments are preferentially ligated to themselves due to proximity and favorable intra-molecular ligation of the single stranded templates shown at D. Double stranded circularization is also acceptable. A non-limiting example of a ligase is CircLigase™, a thermal stable ligase that catalyzes the intramolecular ligation (circularization) of single stranded DNA templates containing 5-prime phosphates and 3-prime hydroxyl groups. Once the circular target molecules are formed, in some cases, single stranded and double stranded linear DNA molecules are degraded through exonuclease digestion, shown at E. The circular molecules, devoid of free or open ends, are resistant to degradation. S1 nuclease is one non-limiting example of exonuclease that will degrade linear DNA molecules.

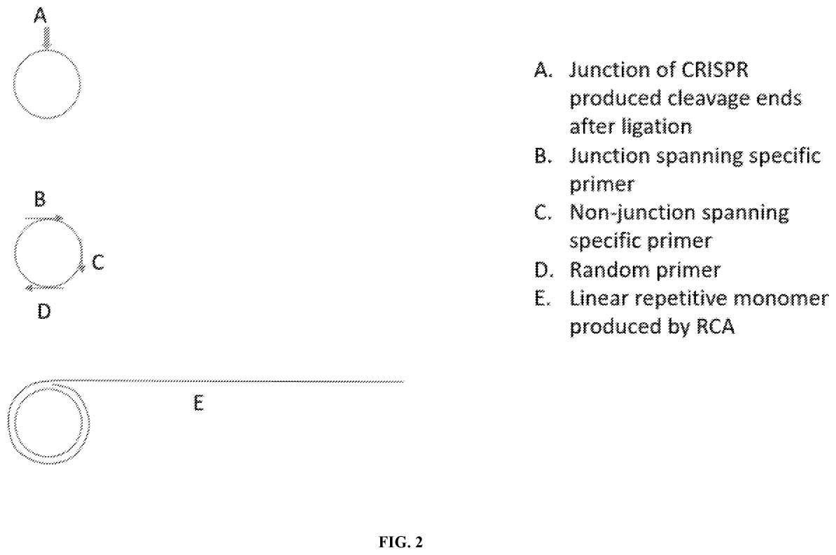

FIG. 2 provides a non-limiting exemplified illustration of the rolling circle amplification step. The circular molecules, having a junction of CRISPR produced cleavage ends after ligation, shown at A, are then subjected to rolling circle amplification (RCA). RCA can be initiated through a random primer (B), a junction spanning locus specific primer (C), or a non junction spanning locus specific primer (D). Random priming will allow for increase amplification through hyper branching using a stand displacing polymerase such as Phi29. The dual CRISPR cutting of gDNA may provide sufficient specificity for random priming RCA. In the event that greater specificity is needed, locus specific RCA primers can be used. These primers may be designed to any sequence within the target circle molecules, however, to avoid amplification of chimeric molecules due to ligation, the specific primers should partially span the junction of the two specific CRISPR sites. For example, the locus specific RCA primers would partially span the 5′ end of the first CRISPR cut location and the 3′ end of the second CRISPR cut location. This will insure only molecules with specific end ligation of the CRISPR cut sites will be preferentially bound by the junction primer. For increased specificity and increased amplification, a combination of specific junction spanning primers AND random primers can be used to employ the strand displacing, branched amplification typical of rolling circle amplification. A linear repetitive monomer is then produced by rolling circle amplification, shown at E.

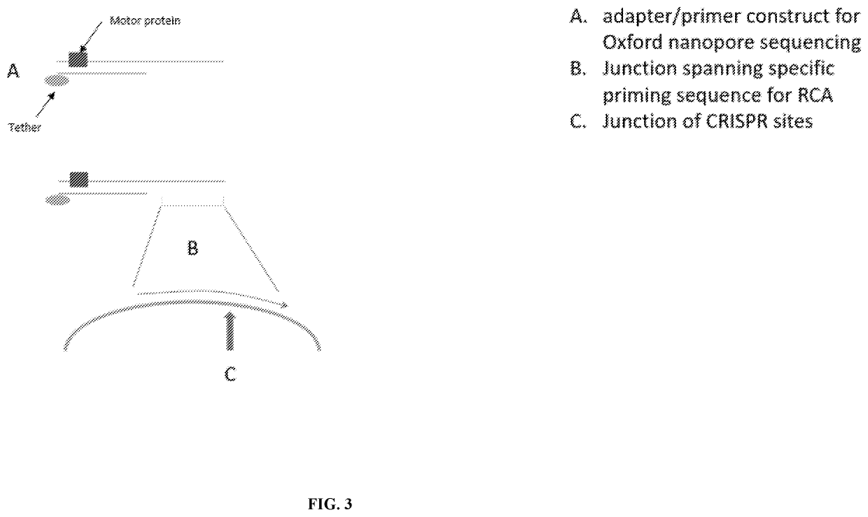

FIG. 3 provides a non-limiting exemplified illustration of the primer and adaptor scheme used in the sequence analysis step. Depending on the sequencing technology used, the 5-prime end of the RCA primers can be “tailed” with adapter sequences specific to the sequencing system being employed. In the case of the Oxford nanopore sequencing devices, such as the deployable MinION, the primers may be synthesized with the motor protein used to initiate nanopore sequencing, shown at A. The primer, in some cases, has a junction spanning sequence for rolling circle amplification (shown at B) spanning the CRISPR sites (shown at C). This approach may enable the real time amplification and nanopore sequencing of consensus circular templates. Because of the nanopore sequencing ability to generate real time data, target molecules of interest are sequenced until the minimum required accuracy of the template is achieved. Molecules may be sampled in real time and discarded once accurate consensus sequencing is achieved or if the molecule being sequenced is not the desired molecule. Each additional molecule is then sampled and sequenced to consensus accuracy. If a target sequence has achieved consensus accuracy threshold, or if the target sequence accuracy was achieved on a previously sequenced molecule, the molecules may be discarded and the run stopped once all targets have achieved the desired sequencing result.

Variations of the technology may include the targeting of multiplexed targets of both RNA and DNA using different guided endonucleases or site-specific restriction enzymes. Circular ligation may also be enabled by the use of a single stranded splint ligation specific to the ends of the CRISPR cleavage sites. The splint may also include barcode sequence, or unique molecule identifiers in the splint. In the latter example, the “gap” corresponding to the barcode sequence may be filled in from the 3′ end of one CRISPR site to the 5′ end of the single stranded molecule, closing the circle with ligation.

As a result of practice of methods disclosed herein, one obtains a library that is both highly amplified, highly representative of the total distribution sites for a mobile element, and highly resistant to error propagation in the synthesis process.

Methods, compositions and kits are provided for sequencing of targeted nucleic acids. These methods, compositions and kits find use in a number of applications, such as point of care detection of time critical genomic information, infectious disease detection in humans, plants and animals in real time and in remote locations, forensic DNA analysis and microbiome detection. These and other objects, advantages, and features of the invention will become apparent to those persons skilled in the art upon reading the details of the compositions and methods as more fully described below.

### Endonuclease for Targeted Cleavage of Nucleic Acid

Methods disclosed herein comprise targeting cleavage of the nucleic acid using a site-specific, targetable, and/or engineered nuclease or nuclease system. Such nucleases may create double-stranded break (DSBs) at desired locations in a genomic DNA, cDNA, or other nucleic acid molecule. In other examples, a nuclease may create a single strand break. In some cases, two nucleases are used, each of which generates a single strand break. Many cleavage enzymes consistent with the disclosure herein share a trait that they yield molecules having an end accessible for single stranded or double stranded exonuclease activity.

The endonuclease used herein can be a restriction enzyme specific to at least one site on the nucleic acid. The endonuclease described herein can be specific to a repetitive nucleic sequence in a host genome, such as a transposon or other repeat, a centromeric region, or other repeat sequence.

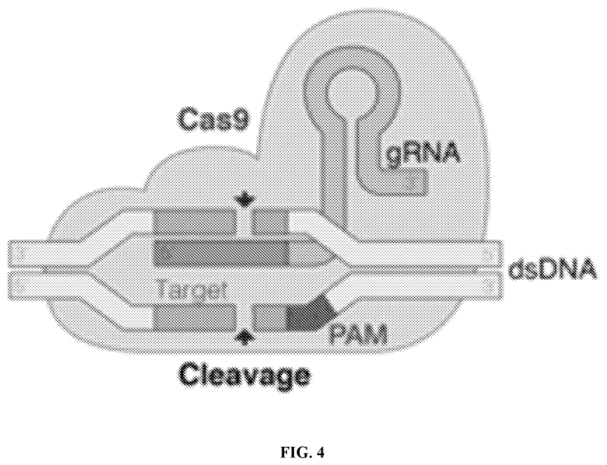

Endonucleases consistent with the disclosure herein variously include at least one selected from Clustered Regulatory Interspaced Short palindromic Repeat (CRISPR)/Cas system protein-gRNA complexes, Zinc Finger Nucleases (ZFN), and Transcription activator like effector nucleases (TALEN). In some embodiments, the gRNAs are complementary to at least one site on the nucleic acid. Other programmable, nucleic acid sequence specific endonucleases are also consistent with the disclosure herein. FIG. 4 shows a CRISPR structure that can be used to selectively cleaving the target nucleic acid.

Engineered nucleases such as zinc finger nucleases (ZFNs), Transcription Activator-Like Effector Nucleases (TALENs), engineered homing endonucleases, and RNA or DNA guided endonucleases, such as CRISPR/Cas such as Cas9 or CPF1, and/or Argonaute systems, are particularly appropriate to carry out some of the methods of the present disclosure. Additionally or alternatively, RNA targeting systems may be used, such as CRISPR/Cas systems including c2c2 nucleases.

For example, some restriction endonucleases consistent with the disclosure herein are Alu specific restriction enzymes. A restriction is Alu specific or, for that matter, other target ‘specific’ if it cuts a target and does not cut other substrates, or cuts other targets infrequently so as to differentially deplete its ‘specific’ target. The presence of a non-Alu or other non-target cleavage, such as due to the rare occurrence of the cleavage site elsewhere in a host genome or transcriptome, or in a pathogen or other rare nucleic acid present in a sample, does not render an endonuclease ‘nonspecific’ so long as differential depletion of undesired nucleic acid is effected. In some embodiments, the nucleic acid comprises at least one sequence that maps to at least one nucleic acid recognition site selected from the group consisting of recognition sites of AluI, AsuHPI, Bpu10I, BssECI, BstDEI, BstMAI, HinfI, and BstTUI.

Methods disclosed herein may comprise cleaving a target nucleic acid using CRISPR systems, such as a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. Alternatively, Class 2 systems include a single effector molecule and include Type II, Type V, and Type VI.

CRISPR systems used in some methods disclosed herein may comprise a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence. CRISPR systems may comprise a single or multiple guiding RNAs. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs.

A gRNA may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some examples, the spacer sequence may be less than 10 or more than 36 nucleotides in length.

A gRNA may comprise a repeat sequence. In some cases, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the spacer sequence may be less than 10 or more than 50 nucleotides in length.

A gRNA may comprise one or more synthetic nucleotides, non-naturally occurring nucleotides, nucleotides with a modification, deoxyribonucleotide, or any combination thereof. Additionally or alternatively, a gRNA may comprise a hairpin, linker region, single stranded region, double stranded region, or any combination thereof. Additionally or alternatively, a gRNA may comprise a signaling or reporter molecule.

A CRISPR nuclease may be endogenously or recombinantly expressed. A CRISPR nuclease may be encoded on a chromosome, extrachromosomally, or on a plasmid, synthetic chromosome, or artificial chromosome. A CRISPR nuclease may be provided as a polypeptide or mRNA encoding the polypeptide. In such examples, polypeptide or mRNA may be delivered through standard mechanisms known in the art, such as through the use of cell permeable peptides, nanoparticles, or viral particles.

gRNAs may be encoded by genetic or episomal DNA. gRNAs may be provided or delivered concomitantly with a CRISPR nuclease or sequentially. Guide RNAs may be chemically synthesized, in vitro transcribed or otherwise generated using standard RNA generation techniques known in the art.

A CRISPR system may be a Type II CRISPR system, for example a Cas9 system. The Type II nuclease may comprise a single effector protein, which, in some cases, comprises a RuvC and HNH nuclease domains. In some cases a functional Type II nuclease may comprise two or more polypeptides, each of which comprises a nuclease domain or fragment thereof. The target nucleic acid sequences may comprise a 3′ protospacer adjacent motif (PAM). In some examples, the PAM may be 5′ of the target nucleic acid. Guide RNAs (gRNA) may comprise a single chimeric gRNA, which contains both crRNA and tracrRNA sequences. Alternatively, the gRNA may comprise a set of two RNAs, for example a crRNA and a tracrRNA. The Type II nuclease may generate a double strand break, which is some cases creates two blunt ends. In some cases, the Type II CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type II nickase is used to generate two single strand breaks, the resulting nucleic acid free ends may either be blunt, have a 3′ overhang, or a 5′ overhang. In some examples, a Type II nuclease may be catalytically dead such that it binds to a target sequence, but does not cleave. For example, a Type II nuclease may have mutations in both the RuvC and HNH domains, thereby rendering the both nuclease domains non-functional. A Type II CRISPR system may be one of three sub-types, namely Type II-A, Type II-B, or Type II-C.

A CRISPR system may be a Type V CRISPR system, for example a Cpf1, C2c1, or C2c3 system. The Type V nuclease may comprise a single effector protein, which in some cases comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some cases, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which in some cases generates a 5′ overhang. In some cases, the Type V CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type V nickase is used to generate two single strand breaks, the resulting nucleic acid free ends may either be blunt, have a 3′ overhang, or a 5′ overhang. In some examples, a Type V nuclease may be catalytically dead such that it binds to a target sequence, but does not cleave. For example, a Type V nuclease could have mutations a RuvC domain, thereby rendering the nuclease domain non-functional.

A CRISPR system may be a Type VI CRISPR system, for example a C2c2 system. A Type VI nuclease may comprise a HEPN domain. In some examples, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some cases, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some examples, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some methods disclosed herein, Argonaute (Ago) systems may be used to cleave target nucleic acid sequences. Ago protein may be derived from a prokaryote, eukaryote, or archaea. The target nucleic acid may be RNA or DNA. A DNA target may be single stranded or double stranded. In some examples, the target nucleic acid does not require a specific target flanking sequence, such as a sequence equivalent to a protospacer adjacent motif or protospacer flanking sequence. The Ago protein may create a double strand break or single strand break. In some examples, when a Ago protein forms a single strand break, two Ago proteins may be used in combination to generate a double strand break. In some examples, an Ago protein comprises one, two, or more nuclease domains. In some examples, an Ago protein comprises one, two, or more catalytic domains. One or more nuclease or catalytic domains may be mutated in the Ago protein, thereby generating a nickase protein capable of generating single strand breaks. In other examples, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In many examples, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the gDNA may be less than 10 nucleotides in length. In some examples, the gDNA may be more than 50 nucleotides in length.

Argonaute-mediated cleavage may generate blunt end, 5′ overhangs, or 3′ overhangs. In some examples, one or more nucleotides are removed from the target site during or following cleavage.

Argonaute protein may be endogenously or recombinantly expressed. Argonaute may be encoded on a chromosome, extrachromosomally, or on a plasmid, synthetic chromosome, or artificial chromosome. Additionally or alternatively, an Argonaute protein may be provided as a polypeptide or mRNA encoding the polypeptide. In such examples, polypeptide or mRNA may be delivered through standard mechanisms known in the art, such as through the use of peptides, nanoparticles, or viral particles.

Guide DNAs may be provided by genetic or episomal DNA. In some examples, gDNA are reverse transcribed from RNA or mRNA. In some examples, guide DNAs may be provided or delivered concomitantly with an Ago protein or sequentially. Guide DNAs may be chemically synthesized, assembled, or otherwise generated using standard DNA generation techniques known in the art. Guide DNAs may be cleaved, released, or otherwise derived from genomic DNA, episomal DNA molecules, isolated nucleic acid molecules, or any other source of nucleic acid molecules.

Nuclease fusion proteins may be recombinantly expressed. A nuclease fusion protein may be encoded on a chromosome, extrachromosomally, or on a plasmid, synthetic chromosome, or artificial chromosome. A nuclease and a chromatin-remodeling enzyme may be engineered separately, and then covalently linked. A nuclease fusion protein may be provided as a polypeptide or mRNA encoding the polypeptide. In such examples, polypeptide or mRNA may be delivered through standard mechanisms known in the art, such as through the use of peptides, nanoparticles, or viral particles.

A guide nucleic acid may complex with a compatible nucleic acid-guided nuclease and may hybridize with a target sequence, thereby directing the nuclease to the target sequence. A subject nucleic acid-guided nuclease capable of complexing with a guide nucleic acid may be referred to as a nucleic acid-guided nuclease that is compatible with the guide nucleic acid. Likewise, a guide nucleic acid capable of complexing with a nucleic acid-guided nuclease may be referred to as a guide nucleic acid that is compatible with the nucleic acid-guided nucleases.

A guide nucleic acid may be DNA. A guide nucleic acid may be RNA. A guide nucleic acid may comprise both DNA and RNA. A guide nucleic acid may comprise modified of non-naturally occurring nucleotides. In cases where the guide nucleic acid comprises RNA, the RNA guide nucleic acid may be encoded by a DNA sequence on a polynucleotide molecule such as a plasmid, linear construct, or editing cassette as disclosed herein.

A guide nucleic acid may comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some aspects, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 nucleotides long. The guide sequence may be 10-25 nucleotides in length. The guide sequence may be 10-20 nucleotides in length. The guide sequence may be 15-30 nucleotides in length. The guide sequence may be 20-30 nucleotides in length. The guide sequence may be 15-25 nucleotides in length. The guide sequence may be 15-20 nucleotides in length. The guide sequence may be 20-25 nucleotides in length. The guide sequence may be 22-25 nucleotides in length. The guide sequence may be 15 nucleotides in length. The guide sequence may be 16 nucleotides in length. The guide sequence may be 17 nucleotides in length. The guide sequence may be 18 nucleotides in length. The guide sequence may be 19 nucleotides in length. The guide sequence may be 20 nucleotides in length. The guide sequence may be 21 nucleotides in length. The guide sequence may be 22 nucleotides in length. The guide sequence may be 23 nucleotides in length. The guide sequence may be 24 nucleotides in length. The guide sequence may be 25 nucleotides in length.

A guide nucleic acid may comprise a scaffold sequence. In general, a “scaffold sequence” includes any sequence that has sufficient sequence to promote formation of a targetable nuclease complex, wherein the targetable nuclease complex comprises a nucleic acid-guided nuclease and a guide nucleic acid comprising a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some aspects, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some aspects, at least one of the two sequence regions is about or more than about 5, 6, 7, 8, 9, 10, 11, 12, 13, 14, 15, 16, 17, 18, 19, 20, 21, 22, 23, 24, 25, 30, 40, 50, or more nucleotides in length. In some aspects, at least one of the two sequence regions is about 10-30 nucleotides in length. At least one of the two sequence regions may be 10-25 nucleotides in length. At least one of the two sequence regions may be 10-20 nucleotides in length. At least one of the two sequence regions may be 15-30 nucleotides in length. At least one of the two sequence regions may be 20-30 nucleotides in length. At least one of the two sequence regions may be 15-25 nucleotides in length. At least one of the two sequence regions may be 15-20 nucleotides in length. At least one of the two sequence regions may be 20-25 nucleotides in length. At least one of the two sequence regions may be 22-25 nucleotides in length. At least one of the two sequence regions may be 15 nucleotides in length. At least one of the two sequence regions may be 16 nucleotides in length. At least one of the two sequence regions may be 17 nucleotides in length. At least one of the two sequence regions may be 18 nucleotides in length. At least one of the two sequence regions may be 19 nucleotides in length. At least one of the two sequence regions may be 20 nucleotides in length. At least one of the two sequence regions may be 21 nucleotides in length. At least one of the two sequence regions may be 22 nucleotides in length. At least one of the two sequence regions may be 23 nucleotides in length. At least one of the two sequence regions may be 24 nucleotides in length. At least one of the two sequence regions may be 25 nucleotides in length.

A scaffold sequence of a subject guide nucleic acid may comprise a secondary structure. A secondary structure may comprise a pseudoknot region. In some example, the compatibility of a guide nucleic acid and nucleic acid-guided nuclease is at least partially determined by sequence within or adjacent to a pseudoknot region of the guide RNA. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In aspects of the disclosure the terms “guide nucleic acid” refers to a polynucleotide comprising 1) a guide sequence capable of hybridizing to a target sequence and 2) a scaffold sequence capable of interacting with or complexing with a nucleic acid-guided nuclease as described herein.

A guide nucleic acid may be compatible with a nucleic acid-guided nuclease when the two elements may form a functional targetable nuclease complex capable of cleaving a target sequence. Often, a compatible scaffold sequence for a compatible guide nucleic acid may be found by scanning sequences adjacent to native nucleic acid-guided nuclease loci. In other words, native nucleic acid-guided nucleases may be encoded on a genome within proximity to a corresponding compatible guide nucleic acid or scaffold sequence.

Nucleic acid-guided nucleases may be compatible with guide nucleic acids that are not found within the nucleases endogenous host. Such orthogonal guide nucleic acids may be determined by empirical testing. Orthogonal guide nucleic acids may come from different bacterial species or be synthetic or otherwise engineered to be non-naturally occurring.

Orthogonal guide nucleic acids that are compatible with a common nucleic acid-guided nuclease may comprise one or more common features. Common features may include sequence outside a pseudoknot region. Common features may include a pseudoknot region. Common features may include a primary sequence or secondary structure.

A guide nucleic acid may be engineered to target a desired target sequence by altering the guide sequence such that the guide sequence is complementary to the target sequence, thereby allowing hybridization between the guide sequence and the target sequence. A guide nucleic acid with an engineered guide sequence may be referred to as an engineered guide nucleic acid. Engineered guide nucleic acids are often non-naturally occurring and are not found in nature.

In some embodiments the guide RNA molecule interferes with sequencing directly, for example by binding the target sequence to prevent nucleic acid polymerization to occur across the bound sequence. In some embodiments the guide RNA molecule works in tandem with a RNA-DNA hybrid binding moiety such as a protein. In some embodiments the guide RNA molecule directs modification of member of the sequencing library to which it may bind, such as methylation, base excision, or cleavage, such that in some embodiments the member of the sequencing library to which it is bound becomes unsuitable for further sequencing reactions. In some embodiments, the guide RNA molecule directs endonucleolytic cleavage of the DNA molecule to which it is bound, for example by a protein having endonuclease activity such as Cas9 protein. Zinc Finger Nucleases (ZFN), Transcription activator like effector nucleases and Clustered Regulatory Interspaced Short palindromic Repeat/Cas based RNA guided DNA nuclease (CRISPR/Cas9), among others, are compatible with some embodiments of the disclosure herein.

A guide RNA molecule comprises sequence that base-pairs with target sequence that is to be removed from sequencing. In some embodiments the base-pairing is complete, while in some embodiments the base pairing is partial or comprises bases that are unpaired along with bases that are paired to non-target sequence.

A guide RNA may comprise a region or regions that form an RNA ‘hairpin’ structure. Such region or regions comprise partially or completely palindromic sequence, such that 5′ and 3′ ends of the region may hybridize to one another to form a double-strand ‘stem’ structure, which in some embodiments is capped by a non-palindromic loop tethering each of the single strands in the double strand loop to one another.

In some embodiments the Guide RNA comprises a stem loop such as a tracrRNA stem loop. A stem loop such as a tracrRNA stem loop may complex with or bind to a nucleic acid endonuclease such as Cas9 DNA endonuclease. Alternately, a stem loop may complex with an endonuclease other than Cas9 or with a nucleic acid modifying enzyme other than an endonuclease, such as a base excision enzyme, a methyltransferase, or an enzyme having other nucleic acid modifying activity that interferes with one or more DNA polymerase enzymes.

The tracrRNA/CRISPR/Endonuclease system was identified as an adaptive immune system in eubacterial and archaeal prokaryotes whereby cells gain resistance to repeated infection by a virus of a known sequence. See, for example, Deltcheva E, Chylinski K, Sharma C M, Gonzales K, Chao Y, Pirzada Z A et al. (2011) “CRISPR RNA maturation by trans-encoded small RNA and host factor RNase III” Nature 471 (7340): 602-7. doi: 10.1038/nature09886. PMC 3070239. PMID 21455174; Terns M P, Terns R M (2011) “CRISPR-based adaptive immune systems” Curr Opin Microbiol 14 (3): 321-7. doi: 10.1016/j.mib.2011.03.005. PMC 3119747. PMID 21531607; Jinek M, Chylinski K, Fonfara I, Hauer M, Doudna J A, Charpentier E (2012) “A Programmable Dual-RNA-Guided DNA Endonuclease in Adaptive Bacterial Immunity” Science 337 (6096): 816-21. doi: 10.1126/science.1225829. PMID 22745249; and Brouns S J (2012) “A swiss army knife of immunity” Science 337 (6096): 808-9. doi: 10.1126/science.1227253. PMID 22904002. The system has been adapted to direct targeted mutagenesis in eukaryotic cells. See, e.g., Wenzhi Jiang, Huanbin Zhou, Honghao Bi, Michael Fromm, Bing Yang, and Donald P. Weeks (2013) “Demonstration of CRISPR/Cas9/sgRNA-mediated targeted gene modification in Arabidopsis, tobacco, sorghum and rice” Nucleic Acids Res. November 2013; 41(20): e188, Published online Aug. 31, 2013. doi: 10.1093/nar/gkt780, and references therein.

As contemplated herein, guide RNA are used in some embodiments to provide sequence specificity to a DNA endonuclease such as a Cas9 endonuclease. In these embodiments a guide RNA comprises a hairpin structure that binds to or is bound by an endonuclease such as Cas9 (other endonucleases are contemplated as alternatives or additions in some embodiments), and a guide RNA further comprises a recognition sequence that binds to or specifically binds to or exclusively binds to a sequence that is to be removed from a sequencing library or a sequencing reaction. The length of the recognition sequence in a guide RNA may vary according to the degree of specificity desired in the sequence elimination process. Short recognition sequences, comprising frequently occurring sequence in the sample or comprising differentially abundant sequence (abundance of AT in an AT-rich genome sample or abundance of GC in a GC-rich genome sample) are likely to identify a relatively large number of sites and therefore to direct frequent nucleic acid modification such as endonuclease activity, base excision, methylation or other activity that interferes with at least one DNA polymerase activity. Long recognition sequences, comprising infrequently occurring sequence in the sample or comprising underrepresented base combinations (abundance of GC in an AT-rich genome sample or abundance of AT in a GC-rich genome sample) are likely to identify a relatively small number of sites and therefore to direct infrequent nucleic acid modification such as endonuclease activity, base excision, methylation or other activity that interferes with at least one DNA polymerase activity. Accordingly, as disclosed herein, in some embodiments one may regulate the frequency of sequence removal from a sequence reaction through modifications to the length or content of the recognition sequence.

Guide RNA may be synthesized through a number of methods consistent with the disclosure herein. Standard synthesis techniques may be used to produce massive quantities of guide RNAs, and/or for highly-repetitive targeted regions, which may require only a few guide RNA molecules to target a multitude of unwanted loci. The double stranded DNA molecules can comprise an RNA site specific binding sequence, a guide RNA sequence for Cas9 protein and a T7 promoter site. In some cases, the double stranded DNA molecules can be less than about 100 bp length. T7 polymerase can be used to create the single stranded RNA molecules, which may include the target RNA sequence and the guide RNA sequence for the Cas9 protein.

Guide RNA sequences may be designed through a number of methods. For example, in some embodiments, non-genic repeat sequences of the human genome are broken up into, for example, 100 bp sliding windows. Double stranded DNA molecules can be synthesized in parallel on a microarray using photolithography.

The windows may vary in size. 30-mer target sequences can be designed with a short trinucleotide protospacer adjacent motif (PAM) sequence of N-G-G flanking the 5′ end of the target design sequence, which in some cases facilitates cleavage. See, among others, Giedrius Gasiunas et al., (2012) “Cas9-crRNA ribonucleoprotein complex mediates specific DNA cleavage for adaptive immunity in bacteria” Proc. Natl. Acad. Sci. USA. September 25, 109(39): E2579-E2586, which is hereby incorporated by reference in its entirety. Redundant sequences can be eliminated and the remaining sequences can be analyzed using a search engine (e.g. BLAST) against the human genome to avoid hybridization against refseq, ENSEMBL and other gene databases to avoid nuclease activity at these sites. The universal Cas9 tracer RNA sequence can be added to the guide RNA target sequence and then flanked by the T7 promoter. The sequences upstream of the T7 promoter site can be synthesized. Due to the highly repetitive nature of the target regions in the human genome, in many embodiments, a relatively small number of guide RNA molecules will digest a larger percentage of NGS library molecules.

Although only about 50% of protein coding genes are estimated to have exons comprising the NGG PAM (photospacer adjacent motif) sequence, multiple strategies are provided herein to increase the percentage of the genome that can be targeted with the Cas9 cutting system. For example, if a PAM sequence is not available in a DNA region, a PAM sequence may be introduced via a combination strategy using a guide RNA coupled with a helper DNA comprising the PAM sequence. The helper DNA can be synthetic and/or single stranded. The PAM sequence in the helper DNA will not be complimentary to the gDNA knockout target in the NGS library, and may therefore be unbound to the target NGS library template, but it can be bound to the guide RNA. The guide RNA can be designed to hybridize to both the target sequence and the helper DNA comprising the PAM sequence to form a hybrid DNA:RNA:DNA complex that can be recognized by the Cas9 system.

The PAM sequence may be represented as a single stranded overhang or a hairpin. The hairpin can, in some cases, comprise modified nucleotides that may optionally be degraded. For example, the hairpin can comprise Uracil, which can be degraded by Uracil DNA Glycosylase.

As an alternative to using a DNA comprising a PAM sequence, modified Cas9 proteins without the need of a PAM sequence or modified Cas9 with lower sensitivity to PAM sequences may be used without the need for a helper DNA sequence.

In further cases, the guide RNA sequence used for Cas9 recognition may be lengthened and inverted at one end to act as a dual cutting system for close cutting at multiple sites. The guide RNA sequence can produce two cuts on a NGS DNA library target. This can be achieved by designing a single guide RNA to alternate strands within a restricted distance. One end of the guide RNA may bind to the forward strand of a double stranded DNA library and the other may bind to the reverse strand. Each end of the guide RNA can comprise the PAM sequence and a Cas9 binding domain. This may result in a dual double stranded cut of the NGS library molecules from the same DNA sequence at a defined distance apart. Some embodiments relate to the generation of guide RNA molecules. Guide RNA molecules are in some cases transcribed from DNA templates. A number of RNA polymerases may be used, such as T7 polymerase, RNA PolI, RNA PolII, RNA PolIII, an organellar RNA polymerase, a viral RNA polymerase, or a eubacterial or archaeal polymerase. In some cases the polymerase is T7.

Guide RNA generating templates comprise a promoter, such as a promoter compatible with transcription directed by T7 polymerase, RNA PolI, RNA PolII, RNA PolIII, an organellar RNA polymerase, a viral RNA polymerase, or a eubacterial or archaeal polymerase. In some cases the promoter is a T7 promoter.

Guide RNA templates encode a tag sequence in some cases. A tag sequence binds to a nucleic acid modifying enzyme such as a methylase, base excision enzyme or an endonuclease. In the context of a larger Guide RNA molecule bound to a nontarget site, a tag sequence tethers an enzyme to a nucleic acid nontarget region, directing activity to the nontarget site. An exemplary tethered enzyme is an endonuclease such as Cas9.

Guide RNA templates are complementary to the nucleic acid corresponding to ribosomal RNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

In many cases, the tag sequence comprises a stem-loop, such as a partial or total stem-loop structure. The ‘stem’ of the stem loop structure is encoded by a palindromic sequence in some cases, either complete or interrupted to introduce at least one ‘kink’ or turn in the stem. The ‘loop’ of the stem loop structure is not involved in stem base pairing in most cases. In some cases, the stem loop is encoded by a tracr sequence, such as a tracr sequence disclosed in references incorporated herein. Some stem loops bind, for example, Cas9 or other endonuclease.

Guide RNA molecules additionally comprise a recognition sequence. The recognition sequence is completely or incompletely reverse-complementary to a nontarget sequence to be eliminated from a nucleic acid library sequence set. As RNA is able to hybridize using base pair combinations (G:U base pairing, for example) that do not occur in DNA-DNA hybrids, the recognition sequence does not need to be an exact reverse complement of the nontarget sequence to bind. In addition, small perturbations from complete base pairing are tolerated in some cases.

### Circularization

Circularization of target nucleic acids can utilize a ligase that enzymatically joins the 5′ end and the 3′ end of the target nucleic acid that has been cleaved by an endonuclease. In some cases, the 5′ end of the target nucleic acid is ligated directly to its 3′ end. Alternatively, the 5′ end of the target nucleic acid is joined to its 3′ end using an adapter, such as a bridge adapter that hybridizes to the 5′ end and the 3′ end of the target nucleic acid.

Any suitable ligase is contemplated to be used to circularize target nucleic acids in methods herein. Exemplary ligases include but are not limited to T7 DNA ligase, T4 DNA ligase, E. coli DNA ligase, CircLigase, T4 RNA ligase 1, T4 RNA ligase 2, Taq DNA libase. Electroligase, SplintR ligase, or combinations thereof.

### Exonuclease Digestion of Non-Target Nucleic Acids

In some cases, an exonuclease is used to digest linear non-target nucleic acids that have not been circularized by earlier steps in the method. For example, the exonuclease may digest linear non-target nucleic acids from a 5′ end to a 3′ end. Alternatively, or in combination, the exonuclease may digest linear non-target nucleic acids from a 3′ end to a 5′ end. Exemplary exonucleases include but are not limited to exonuclease T, exonuclease I, thermolabile exonuclease I, exonuclease III, exonuclease VII, exonuclease VIII, lambda exonuclease, T7 exonuclease, or combinations thereof.

### Primer for Amplification

The amplification step can involve one or more primers. The primer can be selected from random primer, locus specific primer, or combinations thereof.

In some embodiments, two or more primers are used in the amplification step, and the primer can include a first primer, wherein a first primer comprises at least one sequence complementary to at least a portion of the 5′ end and the 3′ end of the target nucleic acid. In some embodiments, two or more primers are used in the amplification step, and the primer can include a first primer and a second primer, wherein a first primer comprises at least one sequence complementary to at least a portion of the 5′ end and the 3′ end of the target nucleic acid, and wherein a second primer comprises a sequence that is complementary to a portion of the target nucleic acid that is not adjacent to the 5′ end or the 3′ end. In some embodiments, the primer contains a sequence of a region of the universal sequence.

The primer can bind to a primer recognition region on the circular nucleic acid. The primer binding sequence can be in the region of the target nucleic acid that is cut by the endonuclease and ligated by the ligase. In some embodiments, the recognition site for the first nicking endonuclease is proximal to the primer binding sequence. In some embodiments, the first primer binding sequence is in the region of the target nucleic acid that is cut by the endonuclease and ligated by the ligase. In one class of embodiments, the recognition site for the first nicking endonuclease is proximal to the first primer binding sequence. In some cases, the primer comprises a barcode or an adapter sequence.

### Amplification and Sequencing

The target nucleic acid may be amplified using any suitable rolling circle amplification method. After a primer is annealed to the circular target nucleic acid, a strand displacing polymerase is used to extend the primer, creating multiple copies of the target nucleic acid. Strand displacing polymerases contemplated for methods herein include, but are not limited to, phi29 polymerase, Bst DNA polymerase, or combinations thereof.

The sequencing of the amplified nucleic acid can be performed concurrently with the step of rolling circle amplification. The sequencing step and the amplification step can both be performed until consensus accuracy (e.g., with a template) is reached.

The nucleic acid amplified according to the method provided herein can be sequenced according to any suitable sequencing methodology, such as direct sequencing, including sequencing by synthesis, sequencing by ligation, sequencing by hybridization, nanopore sequencing and the like. In some embodiments, the immobilized DNA fragments are sequenced on a solid support. In some embodiments, the solid support for sequencing is the same solid support upon which the amplification occurs. In some embodiments, the sequencing is performed using a nanopore based analysis method.

Nanopore-based analysis methods often involve passing a polymeric molecule, for example single-strand DNA (“ssDNA”), through a nanoscopic opening while monitoring a signal such as an electrical signal. Typically, the nanopore is designed to have a size that allows the polymer to pass only in a sequential, single file order. As the polymer molecule passes through the nanopore, differences in the chemical and physical properties of the monomeric units that make up the polymer, for example, the nucleotides that compose the ssDNA, are translated into characteristic electrical signals.

The signal can, for example, be detected as a modulation of the ionic current by the passage of a DNA molecule through the nanopore, which current is created by an applied voltage across the nanopore-bearing membrane or film. Because of structural differences between different nucleotides, different types of nucleotides interrupt the current in different ways, with each different type of nucleotide within the ssDNA producing a type-specific modulation in the current as it passes through a nanopore, and thus allowing the sequence of the DNA to be determined.

Nanopores that have been used for sequencing DNA include protein nanopores held within lipid bilayer membranes, such as α-hemolysin nanopores, and solid state nanopores formed, for example, by ion beam sculpting of a solid-state thin film. Devices using nanopores to sequence DNA and RNA molecules have generally not been capable of reading sequence at a single-nucleotide resolution.

The step of sequencing the amplified nucleic acid can include a) providing a device comprising a substrate having an array of nanopores; each nanopore fluidically connected to an upper fluidic region and a lower fluidic region; wherein each upper fluidic region is fluidically connected through a an upper resistive opening to an upper liquid volume; and each lower fluidic region is connected to a lower liquid volume, and wherein the upper liquid volume and the lower liquid volume are each fluidically connected to two or more fluidic regions, wherein the device comprises an upper drive electrode in the upper liquid volume, a lower drive electrode in the lower liquid volume, and a measurement electrode in either the upper liquid volume or the lower liquid volume; b) placing a polymer molecule to be sequenced into one or more upper fluidic regions; c) applying a voltage across the upper and lower drive electrodes so as to pass a current through the nanopore such that the polymer molecule is translated through the nanopore; d) measuring the current through the nanopore over time; and e) using the measured current over time in step (d) to determine sequence information about the polymer molecule.

Additional sequencing methods include, but are not limited to, massively parallel signature sequencing, polony sequencing, 454 pyrosequencing, Illumina sequencing, combinatorial probe anchor synthesis, SOLiD sequencing, Ion Torrent semiconductor sequencing, DNA nanoball sequencing, Heliscope single molecule sequencing, single molecule real time sequencing, microfluidic sequencing, tunneling currents DNA sequencing, sequencing by hybridization, sequencing with mass spectrometry, RNAP sequencing, and combinations thereof.

Methods described herein can include performing a genetic analysis of the target nucleic acid. Genome sequence databases can be searched to find sequences which are related to the second nucleic acid. The search can generally be performed by using computer-implemented search algorithms to compare the query sequences with sequence information stored in a plurality of databases accessible via a communication network, for example, the Internet. Examples of such algorithms include the Basic Local Alignment Search Tool (BLAST) algorithm, the PSI-blast algorithm, the Smith-Waterman algorithm, the Hidden Markov Model (HMM) algorithm, and other like algorithms.

### DEFINITIONS

A partial list of relevant definitions is as follows.

“Amplified nucleic acid” or “amplified polynucleotide” includes any nucleic acid or polynucleotide molecule whose amount has been increased by any nucleic acid amplification or replication method performed in vitro as compared to its starting amount. For example, an amplified nucleic acid is optionally obtained from a polymerase chain reaction (PCR) which can, in some instances, amplify DNA in an exponential manner (for example, amplification to 2ⁿ copies in n cycles) wherein most products are generated from intermediate templates rather than directly from the sample template. Amplified nucleic acid is alternatively obtained from a linear amplification, where the amount increases linearly over time and which, in some cases, produces products that are synthesized directly from the sample.

“Amplification product” refers to a product resulting from an amplification reaction such as a polymerase chain reaction or a linear amplification.

An “amplicon” is a polynucleotide or nucleic acid that is the source and/or product of natural or artificial amplification or replication events.

The term “biological sample” or “sample” generally refers to a sample or part isolated from a biological entity. The biological sample, in some cases, shows the nature of the whole biological entity and examples include, without limitation, bodily fluids, dissociated tumor specimens, cultured cells, and any combination thereof. Biological samples come from one or more individuals. One or more biological samples come from the same individual. In one non limiting example, a first sample is obtained from an individual's blood and a second sample is obtained from an individual's tumor biopsy. Examples of biological samples include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. In some cases, a blood sample comprises circulating tumor cells or cell free DNA, such as tumor DNA or fetal DNA. The samples include nasopharyngeal wash. Examples of tissue samples of the subject include but are not limited to, connective tissue, muscle tissue, nervous tissue, epithelial tissue, cartilage, cancerous or tumor sample, or bone. Samples are obtained from a human or an animal. Samples are obtained from a mammal, including vertebrates, such as murines, simians, humans, farm animals, sport animals, or pets. Samples are obtained from a living or dead subject. Samples are obtained fresh from a subject or have undergone some form of pre-processing, storage, or transport.

Nucleic acid sample as used herein refers to a nucleic acid sample for which sequence information is to be determined, A nucleic acid sample is extracted from a biological sample above, in some cases. Alternatively, a nucleic acid sample is artificially synthesized, synthetic, or de novo synthesized in some cases. The DNA sample is genomic in some cases, while in alternate cases the DNA sample is derived from a reverse-transcribed RNA sample.

“Bodily fluid” generally describes a fluid or secretion originating from the body of a subject. In some instances, bodily fluid is a mixture of more than one type of bodily fluid mixed together. Some non-limiting examples of bodily fluids include but are not limited to: blood, urine, bone marrow, spinal fluid, pleural fluid, lymphatic fluid, amniotic fluid, ascites, sputum, or a combination thereof.

“Complementary” or “complementarity,” or, in some cases more accurately “reverse-complementarity” refer to nucleic acid molecules that are related by base-pairing. Complementary nucleotides are, generally, A and T (or A and U), or C and G (or G and U). Functionally, two single stranded RNA or DNA molecules are complementary when they form a double-stranded molecule through hydrogen-bond mediated base paring. Two single stranded RNA or DNA molecules are said to be substantially complementary when the nucleotides of one strand, optimally aligned and with appropriate nucleotide insertions or deletions, pair with at least about 90% to about 95% or greater complementarity, and more preferably from about 98% to about 100%) complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions and not stringent hybridization conditions. Hybridization temperatures are generally at least about 2° C. to about 6° C. lower than melting temperatures (Tₘ).

As indicated herein, standard single-letter amino acid residue abbreviations as known in the art are used to refer to the twenty amino acids involved in cellular ribosomally driven polypeptide synthesis.

“Combinatorial labeling” is a method herein by which two or more barcodes are used to label a molecule. The two or more barcodes label a polynucleotide. The barcodes, each, alone in some cases are associated with information. Alternatively, the combination of the barcodes together is associated with information. In some cases a combination of barcodes is used together to determine in a randomly amplified molecule that the amplification occurred from the original sample template and not a synthetic copy of that template. In some cases, the length of one barcode in combination with the sequence of another barcode is used to label a polynucleotide. In some cases, the length of one barcode in combination with the orientation of another barcode is used to label a polynucleotide. In other cases, the sequence of one barcode is used with the orientation of another barcode to label a polynucleotide. In some cases the sequence of a first and a second bar code, in combination with the distance in nucleotides between them, is used to label or to identify a polynucleotide. In some cases the sequence of a first and a second bar code, in combination with the distance in nucleotides between them and the identity of the nucleotides between them, is used to label or to identify a polynucleotide.

“Double-stranded” refers, in some cases, to two polynucleotide strands that have annealed through complementary base-pairing, such as in a reverse-complementary orientation.

“Known oligonucleotide sequence” or “known oligonucleotide” or “known sequence” refers to a polynucleotide sequence that is known. In some cases, a known oligonucleotide sequence corresponds to an oligonucleotide that has been designed, e.g., a universal primer for next generation sequencing platforms (e.g., Illumina, 454), a probe, an adaptor, a tag, a primer, a molecular barcode sequence, an identifier. A known sequence optionally comprises part of a primer. A known oligonucleotide sequence, in some cases, is not actually known by a particular user but is constructively known, for example, by being stored as data accessible by a computer. A known sequence is optionally a trade secret that is actually unknown or a secret to one or more users but is known by the entity who has designed a particular component of the experiment, kit, apparatus or software that the user is using.

“Library” in some cases refers to a collection of nucleic acids. A library optionally contains one or more target fragments. In some instances the target fragments comprise amplified nucleic acids. In other instances, the target fragments comprise nucleic acid that is not amplified. A library optionally contains nucleic acid that has one or more known oligonucleotide sequence(s) added to the 3′ end, the 5′ end or both the 3′ and 5′ end. The library is optionally prepared so that the fragments contain a known oligonucleotide sequence that identifies the source of the library (e.g., a molecular identification barcode identifying a patient or DNA source). In some instances, two or more libraries are pooled to create a library pool. Libraries are optionally generated with other kits and techniques such as transposon mediated labeling, or “tagmentation” as known in the art. Kits are commercially available. One non-limiting example of a kit is the Illumina NEXTERA kit (Illumina, San Diego, Calif.).

“Locus specific” or “loci specific” in some cases refers to one or more loci corresponding to a location in a nucleic acid molecule (e.g., a location within a chromosome or genome). In some instances, a locus is associated with genotype. In some instances, loci are directly isolated and enriched from the sample, e.g., based on hybridization and/or other sequence-based techniques, or alternatively they may are selectively amplified using the sample as a template prior to detection of the sequence. In some instances, loci are selected on the basis of DNA level variation between individuals, based upon specificity for a particular chromosome, based on CG content and/or required amplification conditions of the selected loci, or other characteristics that will be apparent to one skilled in the art upon reading the present disclosure. A locus optionally refers to a specific genomic coordinate or location in a genome as denoted by the reference sequence of that genome.

“Long nucleic acid” refers, in some cases, to a polynucleotide longer than 1, 2, 3, 4, 5, 6, 7, 8, 9, or 10 kilobases.

The term “melting temperature” or “Tₘ” commonly refers to the temperature at which a population of double-stranded nucleic acid molecules becomes half dissociated into single strands. Equations for calculating the Tₘ of nucleic acids are well known in the art. One equation that gives a simple estimate of the Tₘ value is as follows: Tₘ=81.5+16.6(log 10[Na⁺])0.41(%[G+C])−675/n−1.0 m, when a nucleic acid is in aqueous solution having cation concentrations of 0.5 M or less, the (G+C) content is between 30% and 70%, n is the number of bases, and m is the percentage of base pair mismatches (see, e.g., Sambrook J et al., Molecular Cloning, A Laboratory Manual, 3rd Ed., Cold Spring Harbor Laboratory Press (2001)). Other references include more sophisticated computations, which take structural as well as sequence characteristics into account for the calculation of Tₘ.

“Nucleotide” refers to a base-sugar-phosphate combination. Nucleotides are monomeric units of a nucleic acid sequence (e.g., DNA and RNA). The term nucleotide includes naturally and non-naturally occurring ribonucleoside triphosphates ATP, TTP, UTP, CTG, GTP, and ITP, for example and deoxyribonucleoside triphosphates such as dATP, dCTP, dITP, dUTP, dGTP, dTTP, or derivatives thereof. Such derivatives include, for example, [aS]dATP, 7-deaza-dGTP and 7-deaza-dATP, and, for example, nucleotide derivatives that confer nuclease resistance on the nucleic acid molecule containing them. The term nucleotide as used herein also refers to dideoxyribonucleoside triphosphates (ddNTPs) and their derivatives. Illustrative examples of dideoxyribonucleoside triphosphates include, ddATP, ddCTP, ddGTP, ddITP, ddUTP and ddTTP, for example.

“Polymerase” refers to an enzyme that links individual nucleotides together into a strand, using another strand as a template.

“Polymerase chain reaction” or “PCR” refers to a technique for replicating a specific piece of selected DNA in vitro, even in the presence of excess non-specific DNA. Primers are added to the selected DNA, where the primers initiate the copying of the selected DNA using nucleotides and, typically, Taq polymerase or the like. By cycling the temperature, the selected DNA is repetitively denatured and copied. A single copy of the selected DNA, even if mixed in with other, random DNA, in some cases, is amplified to obtain thousands, millions, or billions of replicates. The polymerase chain reaction is used to detect and measure very small amounts of DNA and to create customized pieces of DNA.

The term “polynucleotides” or “nucleic acids” includes but is not limited to various DNA, RNA molecules, derivatives or combination thereof. These include species such as dNTPs, ddNTPs, DNA, RNA, peptide nucleic acids, cDNA, dsDNA, ssDNA, plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA.

A “primer” generally refers to an oligonucleotide used to, e.g., prime nucleotide extension, ligation and/or synthesis, such as in the synthesis step of the polymerase chain reaction or in the primer extension techniques used in certain sequencing reactions. A primer is alternatively used in hybridization techniques as a means to provide complementarity of a locus to a capture oligonucleotide for detection of a specific nucleic acid region.

“Primer extension product” refers to the product resulting from a primer extension reaction using a contiguous polynucleotide as a template, and a complementary or partially complementary primer to the contiguous sequence.

“Sequencing,” “sequence determination,” and the like generally refers to any and all biochemical methods that may be used to determine the order of nucleotide bases in a nucleic acid.

Before the present methods, compositions and kits are described in greater detail, it is to be understood that this invention is not limited to particular method, composition or kit described, as such may, of course, vary. It is also to be understood that the terminology used herein is for the purpose of describing particular embodiments only, and is not intended to be limiting, since the scope of the present invention will be limited only by the appended claims as construed herein. Examples are put forth so as to provide those of ordinary skill in the art with a more complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein are optionally used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method is contemplated to be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

The nucleic acid is first obtained through extraction and purification or from crude samples such as directly from blood, saliva, urine, fecal, tissue, plant, soil or water, etc. In-vitro CRISPR-CAS systems or similar derivatives are used to produce a nick or double stranded cut flanking the genomic region of interest. This step may be applied to one target locus, such as 16s rRNA genomic sequence, or to many known genomic loci simultaneously, such as STRs for forensic analysis. The guide RNA (gRNA) sequences used are preferably further than 30 bp from each other. The genomic material is then heat denatured and the shorter fragments are preferentially ligated to themselves due to proximity and favorable intra-molecular ligation of the single stranded templates. Double stranded circularization is also acceptable. An example of ligase is CircLigase™, a thermal stable ligase that catalyzes the intramolecular ligation (circularization) of single stranded DNA templates containing 5-prime phosphates and 3-prime hydroxyl groups.

Once the circular target molecules are formed, single stranded and double stranded linear DNA molecules are optionally degraded through exonuclease digestion. The circular molecules, devoid of free or open ends, are resistant to degradation. S1 nuclease is one example of exonuclease that will degrade linear DNA molecules. The circular molecules are then subjected to rolling circle amplification (RCA). RCA can be initiated through a random primer or a locus specific primer. Random priming will allow for increase amplification through hyper branching using a stand displacing polymerase such as Phi29. The dual CRISPR cutting of gDNA may provide sufficient specificity for random priming RCA. In the event that greater specificity is needed, locus specific RCA primers can be used. These primers may be designed to any sequence within the target circle molecules, however, to avoid amplification of chimeric molecules due to ligation, the specific primers should partially span the junction of the two specific CRISPR sites. For example, the locus specific RCA primers would partially span the 5′ end of the first CRISPR cut location and the 3′ end of the second CRISPR cut location. This will insure only molecules with specific end ligation of the CRISPR cut sites will be preferentially bound by the junction primer. For increased specificity and increased amplification, a combination of specific junction spanning primers AND random primers can be used to employ the strand displacing, branched amplification typical of rolling circle amplification.

Depending on the sequencing technology used, the 5-prime end of the RCA primers can be “tailed” with adapter sequences specific to the sequencing system being employed. In the case of the Oxford nanopore sequencing devices, such as the deployable MinION, the primers may be synthesized with the motor protein used to initiate nanopore sequencing. This approach may enable the real time amplification and nanopore sequencing of consensus circular templates. Because of the nanopore sequencing ability to generate real time data, target molecules of interest are sequenced until the minimum required accuracy of the template is achieved. Molecules may be sampled in real time and discarded once accurate consensus sequencing is achieved or if the molecule being sequenced is not the desired molecule. Each additional molecule is then sampled and sequenced to consensus accuracy. If a target sequence has achieved consensus accuracy threshold, or if the target sequence accuracy was achieved on a previously sequenced molecule, the molecules may be discarded and the run stopped once all targets have achieved the desired sequencing result.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of determining a nucleic acid sequence, comprising:
(a) contacting a nucleic acid in a sample to an endonuclease to cleave a target nucleic acid;
(b) ligating the target nucleic acid to form a circular target nucleic acid;
(c) hybridizing at least one primer to the circular target nucleic acid;
(d) amplifying the circular target nucleic acid through rolling circle amplification to generate an amplified nucleic acid; and
(e) performing a sequence analysis of the amplified nucleic acid.

2. The method of claim 1, wherein the target nucleic acid comprises at least one of a Clustered Regularly Interspaced Short Palindromic Repeats (CRISPR) sequence, a zinc finger nuclease (ZFN) sequence, and a transcription activator-like effector nucleases (TALENs) sequence.

3. The method of claim 1, wherein the endonuclease is a restriction enzyme specific to at least one site on the nucleic acid.

4. The method of claim 1, wherein the endonuclease comprises at least one selected from Clustered Regulatory Interspaced Short Palindromic Repeat (CRISPR)/Cas system protein-gRNA complex, Zinc Finger Nucleases (ZFN), and Transcription activator like effector nucleases (TALEN).

5. The method of claim 1, wherein the endonuclease is a Clustered Regulatory Interspaced Short Palindromic Repeat (CRISPR)/Cas system protein-gRNA complex, and wherein the gRNAs are complementary to at least one site on the nucleic acid.

6. The method of claim 1, wherein the primer is selected from random primer, locus specific primer, or a combination thereof.

7. The method of claim 1, wherein the endonuclease cleaves the nucleic acid to form a 5′ end or a 3′ end of the target nucleic acid.

8. The method of claim 7, comprising hybridizing a first primer to the circular target nucleic acid, wherein a first primer comprises at least one sequence complementary to at least a portion of the 5′ end and the 3′ end of the target nucleic acid.

9. The method of claim 7, comprising hybridizing a first primer and a second primer to the circular target nucleic acid, wherein a first primer comprises at least one sequence complementary to at least a portion of the 5′ end and the 3′ end of the target nucleic acid, and wherein a second primer comprises a sequence that is complementary to a portion of the target nucleic acid that is not adjacent to the 5′ end or the 3′ end.

10. The method of claim 1, wherein performing a sequence analysis of the amplified nucleic acid comprises performing a nanopore sequencing analysis.

11. The method of claim 10, the primer comprises an adaptor that binds to a motor protein used in the nanopore sequence analysis.

12. The method of claim 1, wherein the amplifying and the sequencing analysis are performed concurrently.

13. The method of claim 1, wherein the amplifying and the sequencing analysis are performed concurrently, and wherein the sequencing analysis is performed until a pre-determined minimum required accuracy of a template is achieved.

14. The method of claim 1, comprising contacting the sample with exonuclease after step (b).

15. The method of claim 14, wherein the exonuclease does not degrade the circular target nucleic acid.

16. The method of claim 1, comprising performing rolling circle amplification in situ on the circular target nucleic acid.

17. The method of claim 15, wherein the exonuclease is S1 nuclease.

18. The method of claim 1, wherein the nucleic acid comprise any one of single stranded DNA, double stranded DNA, single stranded RNA, double stranded RNA, cDNA, synthetic DNA, artificial DNA, and DNA/RNA hybrids.

19. The method of claim 1, comprising sequencing the amplified nucleic acid through a next-generation sequencing method.

20. The method of claim 1, wherein the sample wherein the sample is selected from saliva, blood, plasma, serum, mucous, feces, urine, cerebrospinal fluid (CSF), skin, tissue, and bone.

21. The method of claim 1, wherein ligating the target nucleic acid is performed using a CircLigase™.

22. The method of claim 1, wherein ligating the target nucleic acid is performed using a single stranded splint, wherein the splint comprises sequences complementary to the ends of the target nucleic acid.

23. A method of preparing a nucleic acid for sequencing, comprising
(a) contacting a nucleic acid in a sample to an endonuclease to cleave a target nucleic acid;
(b) ligating the target nucleic acid sequence to form a circular target nucleic acid;
(c) hybridizing at least one primer to the circular target nucleic acid; and
(d) amplifying the circular target nucleic acid through rolling circle amplification.

24. The method of claim 23, wherein the nucleic acid comprises at least one of a Clustered Regularly Interspaced Short Palindromic Repeats (CRISPR) sequence, a zinc finger nuclease (ZFN) sequence, and a transcription activator-like effector nucleases (TALENs) sequence.

25. The method of claim 23, wherein the endonuclease is a restriction enzyme specific to at least one site on the nucleic acid.

26. The method of claim 23, wherein the endonuclease comprises at least one selected from Clustered Regulatory Interspaced Short Palindromic Repeat (CRISPR)/Cas system protein-gRNA complex, Zinc Finger Nucleases (ZFN), and Transcription activator like effector nucleases.

27. The method of claim 23, wherein the endonuclease is a Clustered Regulatory Interspaced Short Palindromic Repeat (CRISPR)/Cas system protein-gRNA complex, and wherein the gRNAs are complementary to at least one site on the nucleic acid.

28. The method of claim 23, wherein the primer is selected from random primer, locus specific primer, or combinations thereof.

29. The method of claim 23, wherein the endonuclease cleaves the nucleic acid to form a 5′ end or a 3′ end of the target nucleic acid.

30. The method of claim 29, comprising hybridizing a first primer to the circular target nucleic acid, wherein a first primer comprises at least one sequence complementary to at least a portion of the 5′ end and the 3′ end of the target nucleic acid.

31. The method of claim 29, comprising hybridizing a first primer and a second primer to the circular target nucleic acid, wherein a first primer comprises at least one sequence complementary to at least a portion of the 5′ end and the 3′ end of the target nucleic acid, and wherein a second primer comprises a sequence that is complementary to a portion of the target nucleic acid that is not adjacent to the 5′ end or the 3′ end.
